(19) United States
(12) Patent Application Publication
(10) Pub. No.: US 2023/0404415 A1
(43) Pub. Date: Dec. 21, 2023

(54) Method, A Device And An Electronic Apparatus For Adaptive Blood Pressure Monitoring And Model Training

(71) Applicant: Hong Kong Centre for Cerebro-Cardiovasculer Health Engineering Limited, Hong Kong (CN)

(72) Inventors: Yuanting ZHANG, Hong Kong (CN); Nan Ji, Hong Kong (CN); So Lee Ngai, Hong Kong (CN)

(21) Appl. No.: 18/070,483

(22) Filed: Nov. 29, 2022

(30) Foreign Application Priority Data
Jun. 17, 2022 (CN) 202210693101.6

(51) Int. Cl. A61B 5/021; A61B 5/00

(52) U.S. Cl. CPC A61B 5/021; A61B 5/7278; A61B 5/7267; A61B 5/4561; A61B 2562/0219; A61B 2562/0247

(57) ABSTRACT

The present invention provides a method, a device, an electronic apparatus and a computer-readable storage medium for adaptive blood pressure monitoring and model training. The invention relates to the technical field of medical monitoring. The method of adaptive blood pressure monitoring comprises: acquiring a radial arterial blood pressure measurement and a hydrostatic pressure of a radial artery; inputting the radial arterial blood pressure measurement and the hydrostatic pressure into a trained blood pressure conversion model, obtaining a brachial arterial blood pressure value according to the blood pressure conversion model by performing the following operations: weighting the hydrostatic pressure according to a conversion function of the blood pressure conversion model to obtain a weighted hydrostatic pressure; determining the brachial arterial blood pressure value according to the radial arterial blood pressure measurement and the weighted hydrostatic pressure. The present invention realizes the conversion of the radial arterial blood pressure measurement to the brachial arterial blood pressure value, and avoids the influence of external factors such as posture of a human arm on the radial arterial blood pressure measurement.

### FIELD OF THE INVENTION

The present invention relates to the field of medical monitoring technology, specifically, the present invention relates to a method, a device, and an electronic apparatus for adaptive blood pressure monitoring and model training.

### BACKGROUND OF THE INVENTION

Hypertension is a disease known as the “silent killer”, which is painless and non-invasive but causes a series of serious complications and damages vital organs of the body, and is closely related to many diseases, especially cardiovascular and cerebrovascular diseases. With the proliferation of patients, the diversity of causes and the expansion of the affected population, daily monitoring of blood pressure is particularly important with the current trend of increasing attention to health care.

Usually, brachial blood pressure is measured in the upper arm area, at the same height as the heart, but the measurement for the upper arm region is not convenient enough to achieve continuous daily monitoring. With the advancement of blood pressure monitoring technology, portable radial artery-based blood pressure measuring devices can meet the daily monitoring needs of users, but the measurements are easily affected by external factors such as arm posture when performing radial artery blood pressure measurement.

### OBJECTS OF THE INVENTION

An object of the present invention is to provide a method, a device, and an electronic apparatus for adaptive blood pressure monitoring and model training.

The above object is met by the combination of features of the main claims; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

Embodiments of the present invention provide a method, a device, and an electronic apparatus for adaptive blood pressure monitoring and model training that can solve the problem of blood pressure measurements in the prior art that are susceptible to the influence of human posture.

In a first main aspect, the present invention provides a method of adaptive blood pressure monitoring, comprising:
acquiring a radial arterial blood pressure measurement and a hydrostatic pressure of a radial artery;
inputting the radial arterial blood pressure measurement and the hydrostatic pressure into a trained blood pressure conversion model, obtaining a brachial arterial blood pressure value according to the blood pressure conversion model by performing the following operations;
weighting the hydrostatic pressure according to a conversion function of the blood pressure conversion model to obtain a weighted hydrostatic pressure;
determining the brachial arterial blood pressure value according to the radial arterial blood pressure measurement and the weighted hydrostatic pressure.

Preferably, determining the brachial artery blood pressure value according to the radial arterial blood pressure measurement and the weighted hydrostatic pressure, comprises:
using a difference between the radial arterial blood pressure measurement and the weighted hydrostatic pressure as the brachial arterial blood pressure value.

Preferably, the hydrostatic pressure of the radial arterial is detected based on the following method:
detecting the hydrostatic pressure of the radial artery by a hydrostatic level sensor; wherein the hydrostatic level sensor comprises at least one of an accelerometer and a gyroscope; the hydrostatic pressure is represented by a height and an angle of an arm of a target subject.

Preferably, the radial arterial blood pressure measurement is detected based on the following methods:
acquiring blood pressure information of the radial artery by a peripheral arterial blood pressure sensor;
performing a signal processing on the blood pressure information to obtain the radial arterial blood pressure measurement.

Preferably, the blood pressure information comprises at least one of a Korotkoff sound signal, a pressure signal, a photoplethysmogram signal, an electrocardiogram signal and an ultrasound signal.

Preferably, the step of performing a signal processing on the blood pressure information comprises at least one of the following:
performing a signal processing on the Korotkoff sound signal by the auscultatory method;
performing a signal processing on the blood pressure information by oscillometric method;
determining a transit time of a pulse wave according to the blood pressure information, performing a signal processing based on the pulse transit time.

In a second main aspect, the present invention provides a method of training a blood pressure conversion model, comprising.
weighting a sample hydrostatic pressure according to an initial conversion function of an initial conversion model to determine a weighted sample hydrostatic pressure;
determining a brachial arterial blood pressure estimated value according to a sample radial arterial blood pressure measurement and the weighted sample hydrostatic pressure;
iteratively updating the initial conversion function, obtaining a trained blood pressure conversion model and a trained conversion function when a mean square value of the brachial arterial blood pressure estimated value satisfies a pre-set convergence condition.

In a third main aspect, the present invention provides a device for adaptive blood pressure monitoring, comprising:
an acquisition module for acquiring a radial arterial blood pressure measurement and a hydrostatic pressure of a radial artery;
a training module for inputting the radial arterial blood pressure measurement and the hydrostatic pressure into a trained blood pressure conversion model, obtaining a brachial arterial blood pressure value according to the blood pressure conversion model by performing the following operations: weighting the hydrostatic pressure according to a conversion function of the blood pressure conversion model to obtain a weighted hydrostatic pressure; determining the brachial arterial blood pressure value according to the radial arterial blood pressure measurement and the weighted hydrostatic pressure.

In a fourth main aspect, the present invention provides a device for training a blood pressure conversion model, comprising:
a weighting module for weighting a sample hydrostatic pressure according to an initial conversion function of an initial conversion model to determine a weighted sample hydrostatic pressure;
a determining module for determining a brachial arterial blood pressure estimated value according to a sample radial arterial blood pressure measurement and the weighted sample hydrostatic pressure;
an updating module for iteratively updating the initial conversion function, obtaining a trained blood pressure conversion model and a trained conversion function when a mean square value of the brachial arterial blood pressure estimated value satisfies a pre-set convergence condition.

In a fifth main aspect, the present invention provides an electronic apparatus, comprising: a memory, a processor and a computer program stored in the memory, wherein the processor executes the computer program to implement the method steps in the first aspect of the embodiment of the present invention.

In a sixth main aspect, the present invention provides a computer readable storage medium in which a computer program is stored, wherein the computer program being executed by a processor to implement the method steps in the first aspect of the embodiment of the present invention.

In a seventh main aspect, the present invention provides a product of computer program, comprising a computer program, wherein the computer program being executed by a processor to implement the method steps in the first aspect of the embodiment of the present invention.

The present invention determines the brachial artery blood pressure value based on the acquired radial artery blood pressure measurement and the hydrostatic pressure of the radial artery by a trained blood pressure conversion model; during the processing of the blood pressure conversion model, the hydrostatic pressure is weighted according to the conversion function, and the brachial artery blood pressure value is determined based on the weighted hydrostatic pressure and the radial artery blood pressure measurement. The present invention realizes the conversion of radial artery blood pressure measurement to brachial artery blood pressure value and avoids the radial artery blood pressure measurement being influenced by external factors such as arm posture. At the same time, the present invention for the radial artery blood pressure and hydrostatic pressure measuring method, different from the prior art for the brachial artery blood pressure measuring method, is suitable for daily blood pressure monitoring, not subject to the human body posture, avoiding the influence of external factors such as human arm posture or height on the measurement. Compared with traditional brachial artery blood pressure measuring method, that requires the removal of thicker clothing on the arm, the present invention allows for continuous daily monitoring of peripheral arterial regions on human body such as the radial artery (e.g., wrist or fingers), enhancing the convenience and accuracy of blood pressure monitoring, improving the user experience.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 shows a schematic diagram illustrating a method of adaptive blood pressure monitoring provided by an embodiment of the present invention;

FIG. 2 shows a flowchart illustrating a method of adaptive blood pressure monitoring provided by an embodiment of the present invention;

FIG. 3 shows a flowchart illustrating a method of training a blood pressure conversion model provided by an embodiment of the present invention;

FIG. 4 shows a schematic diagram illustrating a computational flow of a method of training a blood pressure conversion model provided by embodiments of the present invention; and

FIG. 5 shows a flowchart illustrating a method of adaptive blood pressure monitoring provided by embodiments of the present invention;

FIG. 6 shows a schematic diagram illustrating the structure of a device for adaptive blood pressure monitoring provided by embodiments of the present invention;

FIG. 7 shows a schematic diagram illustrating the structure of a device for training a blood pressure conversion model provided by embodiments of the present invention; and

FIG. 8 shows a schematic diagram illustrating a structure of an electronic apparatus for adaptive blood pressure monitoring provided by an embodiment of the present invention.

### DESCRIPTION OF PREFERRED EMBODIMENTS

Hereinafter, an embodiment of the present invention will be described in detail with reference to the drawings, wherein the same or similar designations from beginning to end indicate the same or similar components or components having the same or similar functions. The embodiment described below by reference to the accompanying drawings are exemplary and are intended only to explain the present invention and are not to be construed as limiting the present invention.

It will be understood by those of skill in the art that the singular forms “one”, “a” and “the” as used herein may also include the plural forms, unless otherwise stated. It should be further understood that the wording “includes” as used in the specification of this application refers to the presence of features, integers, steps, operations, components and/or assemblies, but does not preclude the presence or addition of one or more other features, integers, steps, operations, components, assemblies and/or groups thereof. It should be understood that when an embodiment of this application refers to a component being “connected” or “coupled” to another component, it may be directly connected or coupled to other components, or there may be intermediate components. In addition, “connected” or “coupled” as used herein may include wirelessly connected or wirelessly coupled. The word “and/or” as used herein includes all or any of the units and all combinations of one or more of the associated listed items.

In order to make the purpose, technical solutions and advantages of this application clearer, the following will be described in further detail in conjunction with the accompanying drawings for the implementation of this application.

Despite recent advances in methods of blood pressure monitoring, there are still many issues and challenges with noninvasive wearable blood pressure monitors for daily use. Traditional mercury sphygmomanometers and oscillometric sphygmomanometers are highly restrictive, requiring the subject to remain stationary, with the center of the cuff at the same level with the heart, and not wearing thick clothing. In addition, mercury sphygmomanometers and oscillometric sphygmomanometers provide only snapshot blood pressure measurement and are not suitable for continuous blood pressure monitoring.

It has been found that, in recent years, the emerging cuffless blood pressure monitoring devices based on pulse transit time can provide beat-to-beat blood pressure measurements and can be worn and carried easily for daily monitoring, but they also require the measurement position to be at the same level with the heart, limiting the use in other postures.

The present invention provides a method, a device, and an electronic apparatus for adaptive blood pressure monitoring and model training, which is designed to solve technical problems in prior art such as those described above.

The technical solutions of the present invention and the technical effects resulting from the technical solutions of the present invention are described below through the description of several exemplary embodiments. It should be noted that the following embodiments can be cross-referenced, borrowed or combined with each other, and the descriptions of the same terms, similar features and similar implementation steps, etc. in different embodiments will not be repeated.

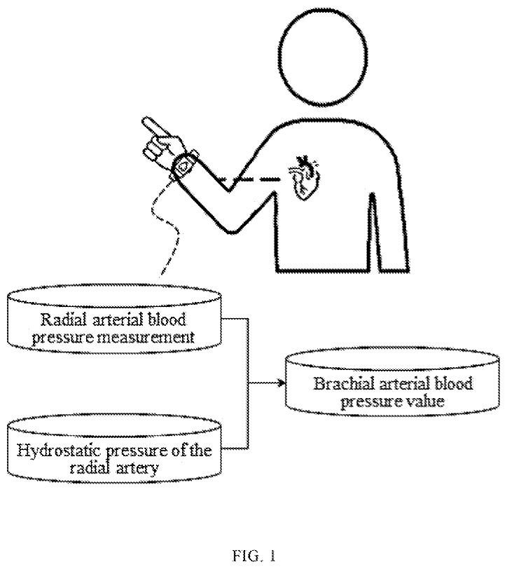

As shown in FIG. 1, the adaptive blood pressure monitoring method of the present invention can be applied in the scenario shown. Specifically, the terminal 101 can acquire a radial arterial blood pressure measurement and a hydrostatic pressure of a radial artery on the wrist or finger of the human body; then the radial artery blood pressure measurement and the hydrostatic pressure are input to a trained blood pressure conversion model, and the brachial artery blood pressure value is obtained through the processing of the blood pressure conversion model, and then the brachial artery blood pressure value is then displayed on the screen of the terminal.

It is understood by a person skilled in the art that the “terminal” used herein may be a smart wearable device such as a watch, a bracelet, a ring, etc.

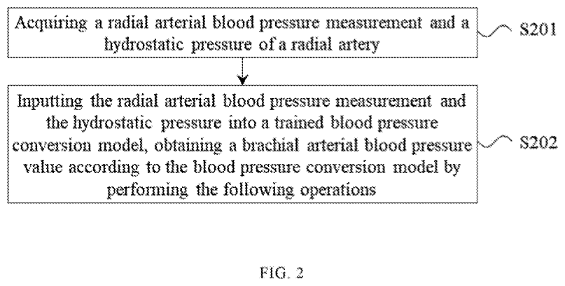

The present invention provides a method of adaptive blood pressure monitoring, as shown in FIG. 2, that can be applied to a server or terminal for adaptive blood pressure monitoring, the method comprising: S201, acquiring a radial arterial blood pressure measurement and a hydrostatic pressure of a radial artery.

Specifically, the server or terminal for adaptive blood pressure monitoring can acquire the radial artery blood pressure measurement and the hydrostatic pressure of the radial artery by a pre-set sensor, wherein the sensors comprise a peripheral arterial blood pressure sensor and a hydrostatic level sensor.

When the method of the present embodiment is applied to a server, the server can interact with the above sensors with information based on a wireless network to obtain the radial artery blood pressure measurement and the hydrostatic pressure of the radial artery.

When the method of the present embodiment is applied to a terminal, the above sensors can be installed with the terminal, and the server can obtain the radial artery blood pressure measurement and the hydrostatic pressure of the radial artery directly through the sensor.

S202, the radial artery blood pressure measurement and the hydrostatic pressure are input to the trained blood pressure conversion model, and the brachial artery blood pressure value is obtained according to the blood pressure conversion model by performing the following operations:
weighting the hydrostatic pressure according to a conversion function of the blood pressure conversion model to obtain a weighted hydrostatic pressure;
determining the brachial arterial blood pressure value according to the radial arterial blood pressure measurement and the weighted hydrostatic pressure.

In the present embodiment, the brachial artery blood pressure value can be calculated by the following equation:

[symbol]

_{HL}(t)=BP(t)−Pₕ(t)* [symbol] (t); (1)

where [symbol] _{HL}(t) is the output brachial artery blood pressure value, BP(t) is the radial artery blood pressure measurement, Pₕ(t) is the hydrostatic blood pressure of the radial artery, and [symbol] (t) is the conversion function of the blood pressure conversion model.

The present invention determines the brachial artery blood pressure value based on the acquired radial artery blood pressure measurement and the hydrostatic pressure of the radial artery by a trained blood pressure conversion model; during the processing of the blood pressure conversion model, the hydrostatic pressure is weighted according to the conversion function, and the brachial artery blood pressure value is determined based on the weighted hydrostatic pressure and the radial artery blood pressure measurement. The present invention realizes the conversion of radial artery blood pressure measurement to brachial artery blood pressure value and avoids the radial artery blood pressure measurement being influenced by external factors such as arm posture. At the same time, the present invention for the radial artery blood pressure and hydrostatic pressure measuring method, different from the prior art for the brachial artery blood pressure measuring method, is suitable for daily blood pressure monitoring, not subject to the human body posture, avoiding the influence of external factors such as human arm posture or height on the measurement. Compared with traditional brachial artery blood pressure measuring method, that requires the removal of thicker clothing on the arm, the present invention allows for continuous daily monitoring of peripheral arterial regions on human body such as the radial artery (e.g., wrist or fingers), enhancing the convenience and accuracy of blood pressure monitoring, improving the user experience.

In an embodiment, determining the brachial arterial blood pressure value according to the radial arterial blood pressure measurement and the weighted hydrostatic pressure comprises using a difference between the radial arterial blood pressure measurement and the weighted hydrostatic pressure as the brachial arterial blood pressure value.

In an embodiment, the hydrostatic pressure of the radial arterial is detected based on the following method: detecting the hydrostatic pressure of the radial artery by a hydrostatic level sensor; wherein the hydrostatic level sensor comprises at least one of an accelerometer and a gyroscope; the hydrostatic pressure is represented by a height and an angle of an arm of a target subject.

In an embodiment of the present invention, the hydrostatic pressure of the radial artery can be monitored based on the hydrostatic level sensor. Since the hydrostatic pressure can be represented by the arm height and angle of the target object, the hydrostatic pressure can be weighted according to the conversion function to calculate the influence of the hydrostatic pressure on the radial artery blood pressure measurement, and then the correction of the radial artery blood pressure measurement based on the hydrostatic blood pressure of the radial artery can be realized to determine the brachial artery blood pressure value, avoiding the influence of arm posture on the radial artery blood pressure measurement.

In an embodiment, the radial arterial blood pressure measurement is detected based on the following methods:
(1) acquiring blood pressure information of the radial artery by a peripheral arterial blood pressure sensor. In an embodiment, the blood pressure information comprises at least one of a Korotkoff sound signal, a pressure signal, a photoplethysmogram signal, an electrocardiogram signal and an ultrasound signal; and
(2) performing a signal processing on the blood pressure information to obtain the radial arterial blood pressure measurement.

In an embodiment, performing a signal processing on the blood pressure information comprises at least one of the following:
a. performing a signal processing on the Korotkoff sound signal by the auscultatory method;
b. performing a signal processing on the blood pressure information by oscillometric method;
c. determining a transit time of a pulse wave according to the blood pressure information, performing a signal processing based on the pulse transit time.

The method provided in the embodiments of the present invention can be applied in a non-invasive continuous blood pressure measurement device, which includes, but is not limited to, a cuff-based blood pressure monitor and a cuffless wearable blood pressure monitor; wherein the cuffless wearable blood pressure monitor can in the format of a watch, a bracelet, a ring, etc.

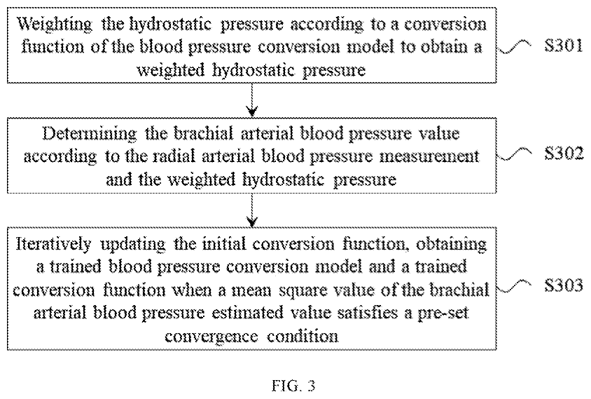

The present invention provides a method training a blood pressure conversion model, as shown in FIG. 3, that can be applied to a server or terminal for performing blood pressure conversion model training, the method comprising:

S301, weighting a sample hydrostatic pressure according to an initial conversion function of an initial conversion model to determine a weighted sample hydrostatic pressure; wherein the weighted sample hydrostatic pressure can indicate the influence of the sample hydrostatic pressure on the radial arterial blood pressure measurement.

In an embodiment, the sample influence [symbol] ₕ(t) can be calculated by the following equation:

[symbol]

ₕ(t)=Pₕ(t)* [symbol] (t); (2)

where, [symbol] (t) is the initial conversion function of the initial conversion model, and Pₕ(t) is the sample hydrostatic pressure of the radial artery.

S302, determining a brachial arterial blood pressure estimated value according to a sample radial arterial blood pressure measurement and the weighted sample hydrostatic pressure.

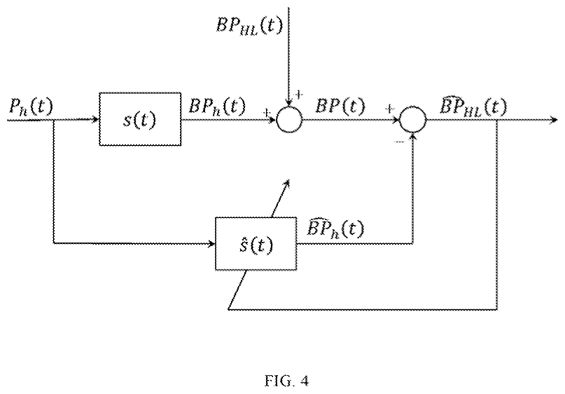

Specifically, as shown in FIG. 4, the server or terminal used to perform blood pressure conversion model training can calculate the brachial artery blood pressure value [symbol] _{HL}(t) based on the following methods:

(1) determining a radial artery blood pressure measurement BP(t) according to the blood pressure information of the radial artery; wherein the blood pressure information comprises at least one of a Korotkoff sound signal, a pressure signal, a photoplethysmogram signal, an electrocardiogram signal and an ultrasound signal. The radial artery blood pressure measurement is influenced by two factors: the actual value of brachial artery blood pressure and the actual influence of the sample hydrostatic pressure:

BP(t)=BPₕ(t)+BP_{HL}(t)=Pₕ(t)*s(t)+BP_{HL}(t); (3)

wherein, s(t) is the actual conversion function of hydrostatic pressure, BP_{HL}(t) is the actual value of brachial blood pressure, and BPₕ(t) is the actual influence of sample hydrostatic pressure.

(2) determining a brachial blood pressure value [symbol] _{HL}(t) according to the radial artery blood pressure measurement BP(t) and the sample influence [symbol] ₕ(t):

[symbol]

_{HL}(t)=BP(t)− [symbol] ₕ(t)=BPₕ(t)+BP_{HL}(t)− [symbol] ₕ(t) (4)

S303, iteratively updating the initial conversion function, obtaining a trained blood pressure conversion model and a trained conversion function when a mean square value of the brachial arterial blood pressure estimated value satisfies a pre-set convergence condition.

Specifically, the mean square value of the estimated brachial artery blood pressure value can be used as the loss function to train the initial conversion model. The equation of the loss function is as follows:

$$\begin{matrix}E [ {HL}_{} {( t )}^{2} ] = E [ \begin{matrix}{{BP}_{HL} ( t )}^{2} + {( {BP}_{h} ( t ) - ( t ) )}^{2} + 2 \cdot \\ {BP}_{HL} ( t ) \cdot ( {BP}_{h} ( t ) - ( t ) )\end{matrix} ] \\ = E [ {{BP}_{HL} ( t )}^{2} ] + E [ {( {BP}_{h} ( t ) - ( t ) )}^{2} ] + \\ E [ 2 \cdot {BP}_{HL} ( t ) \cdot ( {BP}_{h} ( t ) - ( t ) ) ] \\ = E [ {{BP}_{HL} ( t )}^{2} ] + E [ {( {BP}_{h} ( t ) - ( t ) )}^{2} ]\end{matrix} \qquad ( 5 )$$

wherein Pₕ(t) is the input, the signal [BP_{HL}²] is not affected when [symbol] (t) is adjusted so that the loss value of the above loss function is minimized, therefore:

min E[ [symbol] _{HL}²]=E[BP_{HL}²]+min E[(BPₕ− [symbol] ₕ)²] (6)

The update of [symbol] (t) can be calculated according to the partial derivatives of the loss function; the conversion function is adjusted according to the update of [symbol] (t), and E[(BPₕ− [symbol] ₕ)²] is the smallest when the conversion function [symbol] (t) is adjusted so that E[ [symbol] _{HL}²] is the smallest, providing the least squares estimate of BPₕ(t), where the loss function converges and the trained BP conversion model can be determined according to the adjusted conversion function.

This embodiment determines the influence of the sample hydrostatic pressure on the radial artery blood pressure by weighting the sample hydrostatic pressure of the radial artery according to the initial conversion function of the initial conversion model, followed by determining the brachial artery blood pressure value based on the radial artery blood pressure measurement and the influence of the sample hydrostatic pressure; and then iteratively updating the parameters of the initial conversion function, this embodiment achieves iterative training of the initial conversion function when the loss function converges, the trained blood pressure conversion model is obtained. The present invention can determine the loss function based on the mean square value of the estimated brachial blood pressure value, which improves the accuracy and robustness of the blood pressure conversion model and effectively improves the efficiency of the brachial blood pressure conversion.

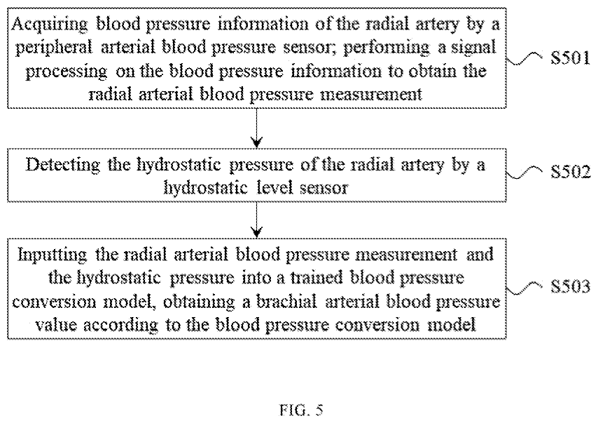

To better understand the above method of adaptive blood pressure monitoring, an example of the present method is detailed below in conjunction with FIG. 5, which comprises the steps of:

S501, acquiring blood pressure information of the radial artery by a peripheral arterial blood pressure sensor; performing a signal processing on the blood pressure information to obtain the radial arterial blood pressure measurement.

S502, detecting the hydrostatic pressure of the radial artery by a hydrostatic level sensor; wherein the hydrostatic level sensor comprises at least one of an accelerometer and a gyroscope; the hydrostatic pressure is represented by a height and an angle of an arm of a target subject.

S503, inputting the radial arterial blood pressure measurement and the hydrostatic pressure into a trained blood pressure conversion model, obtaining a brachial arterial blood pressure value according to the blood pressure conversion model by performing the following operations;

weighting the hydrostatic pressure according to a conversion function of the blood pressure conversion model to obtain a weighted hydrostatic pressure; determining the brachial arterial blood pressure value according to the radial arterial blood pressure measurement and the weighted hydrostatic pressure; wherein, the blood pressure conversion model is obtained based on training as follows:
(1) obtaining a training set, wherein the training set includes sample radial artery blood pressure measurements and sample hydrostatic pressures of the radial artery.
(2) weighting the hydrostatic pressure according to a conversion function of the blood pressure conversion model to obtain a weighted hydrostatic pressure;
(3) determining the brachial arterial blood pressure value according to the radial arterial blood pressure measurement and the weighted hydrostatic pressure.
(4) iteratively updating the initial conversion function, obtaining a trained blood pressure conversion model and a trained conversion function when a mean square value of the brachial arterial blood pressure estimated value satisfies a pre-set convergence condition.

The present invention determines the brachial artery blood pressure value based on the acquired radial artery blood pressure measurement and the hydrostatic pressure of the radial artery by a trained blood pressure conversion model; during the processing of the blood pressure conversion model, the hydrostatic pressure is weighted according to the conversion function, and the brachial artery blood pressure value is determined based on the weighted hydrostatic pressure and the radial artery blood pressure measurement. The present invention realizes the conversion of radial artery blood pressure measurement to brachial artery blood pressure value and avoids the radial artery blood pressure measurement being influenced by external factors such as arm posture. At the same time, the present invention for the radial artery blood pressure and hydrostatic pressure measuring method, different from the prior art for the brachial artery blood pressure measuring method, is suitable for daily blood pressure monitoring, not subject to the human body posture, avoiding the influence of external factors such as human arm posture or height on the measurement. Compared with traditional brachial artery blood pressure measuring method, that requires the removal of thicker clothing on the arm, the present invention allows for continuous daily monitoring of peripheral arterial regions on human body such as the radial artery (e.g., wrist or fingers), enhancing the convenience and accuracy of blood pressure monitoring, improving the user experience.

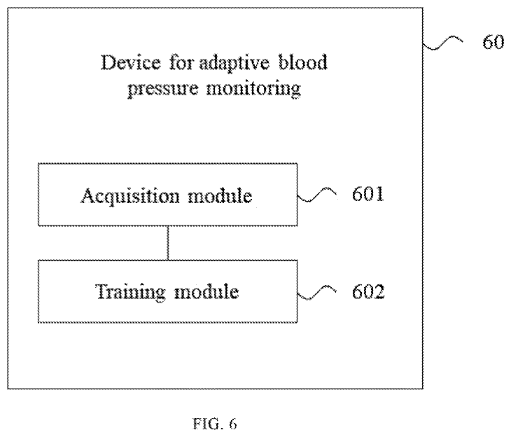

The present invention provides a device for adaptive blood pressure monitoring, as shown in FIG. 6. The device for adaptive blood pressure monitoring 60 comprises: an acquisition module 601 and a training module 602; wherein the acquisition module 601, for acquiring the radial artery blood pressure measurement and the hydrostatic pressure of the radial artery; the training module 602, for inputting the radial arterial blood pressure measurement and the hydrostatic pressure into a trained blood pressure conversion model, obtaining a brachial arterial blood pressure value according to the blood pressure conversion model by performing the following operations: weighting the hydrostatic pressure according to a conversion function of the blood pressure conversion model to obtain a weighted hydrostatic pressure; determining the brachial arterial blood pressure value according to the radial arterial blood pressure measurement and the weighted hydrostatic pressure.

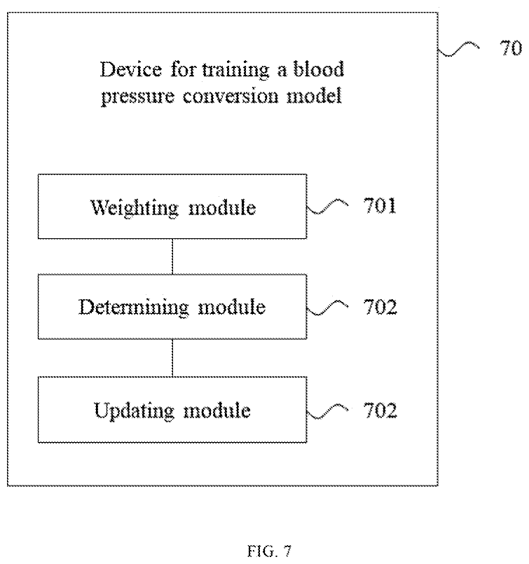

The present invention provides a device for training a blood pressure conversion model, as shown in FIG. 7, the device for training a blood pressure conversion model 70 comprises: a weighting module 701, a determining module 702 and an updating module 703; wherein the weighting module 701, for weighting a sample hydrostatic pressure according to an initial conversion function of an initial conversion model to determine a weighted sample hydrostatic pressure; the determining module 702, for determining a brachial arterial blood pressure estimated value according to a sample radial arterial blood pressure measurement and the weighted sample hydrostatic pressure; the updating module 703, for iteratively updating the initial conversion function, obtaining a trained blood pressure conversion model and a trained conversion function when a mean square value of the brachial arterial blood pressure estimated value satisfies a pre-set convergence condition.

The device of the present embodiment can perform the method provided in the present embodiment with similar implementation principles, and the actions performed by the modules in the device of the present embodiments are corresponding to the steps in the method of the present embodiments, and the detailed functional descriptions of the modules of the device can be specifically referred to the descriptions in the corresponding methods shown in the previous section, which will not be repeated here.

The present invention determines the brachial artery blood pressure value based on the acquired radial artery blood pressure measurement and the hydrostatic pressure of the radial artery by a trained blood pressure conversion model; during the processing of the blood pressure conversion model, the hydrostatic pressure is weighted according to the conversion function, and the brachial artery blood pressure value is determined based on the weighted hydrostatic pressure and the radial artery blood pressure measurement. The present invention realizes the conversion of radial artery blood pressure measurement to brachial artery blood pressure value and avoids the radial artery blood pressure measurement being influenced by external factors such as arm posture. At the same time, the present invention for the radial artery blood pressure and hydrostatic pressure measuring method, different from the prior art for the brachial artery blood pressure measuring method, is suitable for daily blood pressure monitoring, not subject to the human body posture, avoiding the influence of external factors such as human arm posture or height on the measurement. Compared with traditional brachial artery blood pressure measuring method, that requires the removal of thicker clothing on the arm, the present invention allows for continuous daily monitoring of peripheral arterial regions on human body such as the radial artery (e.g., wrist or fingers), enhancing the convenience and accuracy of blood pressure monitoring, improving the user experience.

The present invention provides an electronic apparatus, comprising: a memory, a processor and a computer program stored in the memory, wherein the processor executes the computer program to implement the method steps in the embodiment of the present invention. Compared with the prior art for the brachial artery blood pressure measuring method, the present invention determines the brachial artery blood pressure value based on the acquired radial artery blood pressure measurement and the hydrostatic pressure of the radial artery by a trained blood pressure conversion model; during the processing of the blood pressure conversion model, the hydrostatic pressure is weighted according to the conversion function, and the brachial artery blood pressure value is determined based on the weighted hydrostatic pressure and the radial artery blood pressure measurement. The present invention realizes the conversion of radial artery blood pressure measurement to brachial artery blood pressure value and avoids the radial artery blood pressure measurement being influenced by external factors such as arm posture. At the same time, the present invention for the radial artery blood pressure and hydrostatic pressure measuring method, different from the prior art for the brachial artery blood pressure measuring method, is suitable for daily blood pressure monitoring, not subject to the human body posture, avoiding the influence of external factors such as human arm posture or height on the measurement. Compared with traditional brachial artery blood pressure measuring method, that requires the removal of thicker clothing on the arm, the present invention allows for continuous daily monitoring of peripheral arterial regions on human body such as the radial artery (e.g., wrist or fingers), enhancing the convenience and accuracy of blood pressure monitoring, improving the user experience.

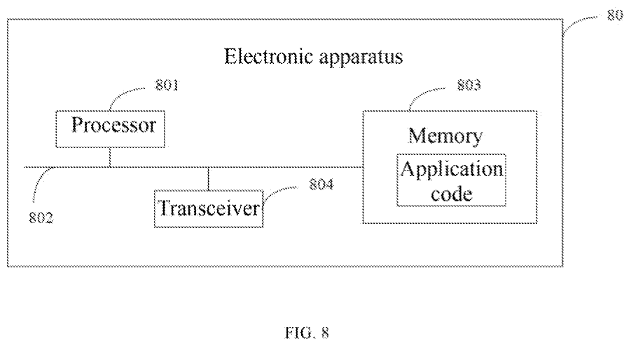

In an optional embodiment, an electronic device is provided, as shown in FIG. 8, the electronic device 800 comprises: a processor 801 and a memory 803, wherein the processor 801 and the memory 803 are connected, e.g., via a bus 802. Optionally, the electronic device 800 may also include a transceiver 804. It is noted that the transceiver 804 is not limited to one in practical applications, and the structure of the electronic device 800 does not constitute a limitation of an embodiment of this application.

The processor 801 may be a CPU (Central Processing Unit), a general-purpose processor, a DSP (Digital Signal Processor), an ASIC (Application Specific Integrated Circuit), an FPGA (FPGA), or a FPC (Fibre Optic Integrated Circuit). Integrated Circuit), FPGA (Field Programmable Gate Array) or other programmable logic device, transistorized logic device, hardware component, or any combination thereof. It may implement or execute various exemplary logic boxes, modules, and circuits described in conjunction with the disclosure of this application. Processor 801 may also be a combination that implements computing function, such as a combination containing one or more microprocessors, a combination of a DSP and a microprocessor, etc.

The bus 802 may include a pathway to transfer information between above components. Bus 802 may be a PCI (Peripheral Component Interconnect) bus or an EISA (Extended Industry Standard Architecture) bus, for example. Bus 802 can be divided into address bus, data bus, control bus, etc. For the convenience of representation, only one thick line is used in FIG. 8, but it does not mean that there is only one bus or one type of bus.

Memory 803 can be ROM (Read Only Memory) or other types of static storage devices that can store static information and instructions, RAM (Random Access Memory) or other types of dynamic storage devices that can store information and instructions, or EEPROM (EEPROM (Electrically Erasable Programmable Read Only Memory), CD-ROM (Compact Disc Read Only Memory) or other optical disc storage, optical disc storage (including compressed disc, laser disc, optical disc, digital universal CD-ROM (Compact Disc Read Only Memory, read-only CD-ROM) or other optical disc storage, optical disc storage (including compact disc, laser disc, optical disk, digital universal disc, Blu-ray disc, etc.), disk storage media or other magnetic storage devices, or any other media capable of carrying or storing desired program code in the form of instructions or data structures and capable of being accessed by a computer, but not limited thereof.

Memory 803 is used to store the application program code for executing the present invention solution and is controlled for execution by processor 801. Processor 801 is used to execute the application program code stored in memory 803 to implement the method steps in the embodiment of the present invention.

The electronic apparatus includes, but are not limited to: smart wearable devices such as cuff-based wearable devices, watches, bracelets, rings, etc.

The present invention provides a computer readable storage medium in which a computer program is stored, wherein the computer program being executed by a processor to implement the method steps in the embodiment of the present invention.

The present invention provides a computer program product or computer program, the computer program product or computer program comprise computer instructions, which being stored in the computer readable storage medium. A processor of the computer device reads the computer instructions from the computer readable storage medium, and the processor executes the computer instructions such that the computer device, implement the following steps:
acquiring a radial arterial blood pressure measurement and a hydrostatic pressure of a radial artery;
inputting the radial arterial blood pressure measurement and the hydrostatic pressure into a trained blood pressure conversion model, obtaining a brachial arterial blood pressure value according to the blood pressure conversion model by performing the following operations;
weighting the hydrostatic pressure according to a conversion function of the blood pressure conversion model to obtain a weighted hydrostatic pressure;
determining the brachial arterial blood pressure value according to the radial arterial blood pressure measurement and the weighted hydrostatic pressure.

The terms “first,” “second,” “third,” “fourth”, “1”, “2”, etc. (if any) in the specification and claims of this application and the accompanying drawings, are used to distinguish similar objects and not to describe a particular order or sequence. It should be understood that the number used is interchangeable where appropriate so that embodiments of the present invention can be implemented in an order other than that illustrated or described here.

It should be understood that although the individual steps in the flowchart of the accompanying drawings are shown sequentially as indicated by the arrows, the steps are not necessarily executed sequentially in the order indicated by the arrows. Unless being stated explicitly herein, in some application scenarios of embodiments of the present invention, the implementation steps in the respective flowcharts may be performed in other orders as needed. In addition, some or all of the steps in each flowchart may include multiple sub-steps or multiple phases based on actual application scenarios. Some or all of these sub-steps or phases may be executed at the same moment, and each of these sub-steps or phases may also be executed separately at different moments. In scenarios where the execution time is different, the order of execution of these sub-steps or phases can be flexibly configured as needed, and this embodiment is not limited in this regard.

The above description is only an optional implementation of part of the application scenario of the present invention. It should be noted that for a person with skill in the art, other similar means of implementation based on the technical principle of the present invention, and without deviating from the technical concept of the present invention, also fall within the scope of protection of the present invention.

We claim:

1. A method of adaptive blood pressure monitoring, comprising:
acquiring a radial arterial blood pressure measurement and a hydrostatic pressure of a radial artery;
inputting the radial arterial blood pressure measurement and the hydrostatic pressure into a trained blood pressure conversion model, obtaining a brachial arterial blood pressure value according to the blood pressure conversion model by performing the following operations;
weighting the hydrostatic pressure according to a conversion function of the blood pressure conversion model to obtain a weighted hydrostatic pressure;
determining the brachial arterial blood pressure value according to the radial arterial blood pressure measurement and the weighted hydrostatic pressure.

2. The method according to claim 1, wherein the step of determining the brachial arterial blood pressure value according to the radial arterial blood pressure measurement and the weighted hydrostatic pressure, comprises:
using a difference between the radial arterial blood pressure measurement and the weighted hydrostatic pressure as the brachial arterial blood pressure value.

3. The method according to claim 1, wherein the hydrostatic pressure of the radial arterial is detected based on the following method:
detecting the hydrostatic pressure of the radial artery by a hydrostatic pressure sensor; wherein the hydrostatic pressure sensor comprises at least one of an accelerometer and a gyroscope; the hydrostatic pressure is represented by a height and an angle of an arm of a target subject.

4. The method according to claim 1, wherein the radial arterial blood pressure measurement is detected based on the following methods:
acquiring blood pressure information of the radial artery by a peripheral arterial blood pressure sensor;
performing a signal processing on the blood pressure information to obtain the radial arterial blood pressure measurement.

5. The method according to claim 4, wherein the blood pressure information comprises at least one of a Korotkoff sound signal, a pressure signal, a photoplethysmogram signal, an electrocardiogram signal and an ultrasound signal.

6. The method according to claim 5, wherein the step of performing a signal processing on the blood pressure information comprises at least one of the following:
performing a signal processing on the Korotkoff sound signal by the auscultatory method;
performing a signal processing on the blood pressure information by oscillometric method;
determining a transit time of a pulse wave according to the blood pressure information, performing a signal processing based on the pulse transit time.

7. A method of training a blood pressure conversion model, comprising:
weighting a sample hydrostatic pressure according to an initial conversion function of an initial conversion model to determine a weighted sample hydrostatic pressure;
determining a brachial arterial blood pressure estimated value according to a sample radial arterial blood pressure measurement and the weighted sample hydrostatic pressure;
iteratively updating the initial conversion function, obtaining a trained blood pressure conversion model and a trained conversion function when a mean square value of the brachial arterial blood pressure estimated value satisfies a pre-set convergence condition.

8. A device for adaptive blood pressure monitoring, comprising:
an acquiring module for acquiring a radial arterial blood pressure measurement and a hydrostatic pressure of a radial artery;
a training module for inputting the radial arterial blood pressure measurement and the hydrostatic pressure into a trained blood pressure conversion model, obtaining a brachial arterial blood pressure value according to the blood pressure conversion model by performing the following operations: weighting the hydrostatic pressure according to a conversion function of the blood pressure conversion model to obtain a weighted hydrostatic pressure; determining the brachial arterial blood pressure value according to the radial arterial blood pressure measurement and the weighted hydrostatic pressure.

9. A device for training a blood pressure conversion model, comprising:
a weighting module for weighting a sample hydrostatic pressure according to an initial conversion function of an initial conversion model to determine a weighted sample hydrostatic pressure;
a determining module for determining a brachial arterial blood pressure estimated value according to a sample radial arterial blood pressure measurement and the weighted sample hydrostatic pressure;
an updating module for iteratively updating the initial conversion function, obtaining a trained blood pressure conversion model and a trained conversion function when a mean square value of the brachial arterial blood pressure estimated value satisfies a pre-set convergence condition.
